(19) United States
(12) Patent Application Publication
(10) Pub. No.: US 2020/0378641 A1
(43) Pub. Date: Dec. 3, 2020

(54) TECHNIQUES FOR DETECTING OR CONSIDERING HABITAT AIR QUALITY

(71) Applicant: AIR DISTRIBUTION TECHNOLOGIES IP, LLC, Glendale, WI (US)

(72) Inventors: Anthony Steven URIARTE, Muskegon, MI (US); Peter SCHMITZ, Byron Center, MI (US)

(21) Appl. No.: 16/584,485

(22) Filed: Sep. 26, 2019

Related U.S. Application Data
(60) Provisional application No. 62/854,752, filed on May 30, 2019.

(51) Int. Cl. F24F 11/30; G01N 33/00; F24F 11/58; F24F 11/65; F24F 11/80; F24F 11/61; G05B 15/02

(52) U.S. Cl. CPC F24F 11/30; G01N 33/0047; G01N 33/004; F24F 11/58; F24F 2110/10; F24F 11/80; F24F 11/61; G05B 15/02; F24F 11/65

(57) ABSTRACT

Examples described herein generally relate to measuring, via one or more sensors, one or more values of air quality parameters in a zone of the habitat, receiving, from one or more other devices, one or more additional values of air quality parameters in other zones of the habitat, determining, based at least in part on the one or more values and the one or more additional values, an air quality condition, and reporting, to a controlling device, one or more instructions based on the air quality condition.

### CLAIM OF PRIORITY UNDER 35 U.S.C. § 119

The present application for patent claims priority to Provisional Application No. 62/854,752, entitled “TECHNIQUES FOR DETECTING OR CONSIDERING HABITAT AIR QUALITY” filed May 30, 2019, which is assigned to the assignee hereof and hereby expressly incorporated by reference herein for all purposes.

### BACKGROUND

People spend much of their time in indoor habitats, seemingly safe from outdoor air quality concerns. Outdoor air quality concerns, however, can become prevalent indoors as well, especially in environments having heating, ventilation, and air conditioning (HVAC) systems that can circulate air throughout an indoor habitat. In some cases, outdoor air quality particles, such as pollen, and/or other harmful indoor substances, such as carbon monoxide, can creep into HVAC ducts and be spread throughout the indoor environment. This may be especially harmful to individual with asthma or other lung associated health issues. Detection mechanisms exist for detecting threshold amounts of harmful air quality products, but may not be adequate for all desired detection levels, and are limited in notification/alarming of detection. An example of a detection mechanism can include a smoke or carbon monoxide detector that is capable of detecting smoke or carbon monoxide, and sounding an alarm when a threshold amount of smoke or carbon monoxide is detected.

### SUMMARY

The following presents a simplified summary of one or more examples in order to provide a basic understanding of such examples. This summary is not an extensive overview of all contemplated examples, and is intended to neither identify key or critical elements of all examples nor delineate the scope of any or all examples. Its sole purpose is to present some concepts of one or more examples in a simplified form as a prelude to the more detailed description that is presented later.

In an example, a device for reporting air quality parameters for a habitat is provided. The device includes one or more sensors configured to measure one or more values of air quality parameters in a zone of the habitat, a transceiver configured to receive, from one or more other devices, one or more additional values of air quality parameters in other zones of the habitat, and at least one processor. The at least one processor is configured to determine, based at least in part on the one or more values and the one or more additional values, an air quality condition, and report, to a controlling device and via the transceiver, one or more instructions based on the air quality condition.

In another example, a computer-implemented method for reporting air quality parameters for a habitat is provided. The method includes measuring, via one or more sensors, one or more values of air quality parameters in a zone of the habitat, receiving, from one or more other devices, one or more additional values of air quality parameters in other zones of the habitat, determining, based at least in part on the one or more values and the one or more additional values, an air quality condition, and reporting, to a controlling device, one or more instructions based on the air quality condition.

In another example, a non-transitory computer-readable medium including code executable by one or more processors for reporting air quality parameters is provided. The code includes code for measuring, via one or more sensors, one or more values of air quality parameters in a zone of the habitat, receiving, from one or more other devices, one or more additional values of air quality parameters in other zones of the habitat, determining, based at least in part on the one or more values and the one or more additional values, an air quality condition, and reporting, to a controlling device, one or more instructions based on the air quality condition.

To the accomplishment of the foregoing and related ends, the one or more examples comprise the features hereinafter fully described and particularly pointed out in the claims. The following description and the annexed drawings set forth in detail certain illustrative features of the one or more examples. These features are indicative, however, of but a few of the various ways in which the principles of various examples may be employed, and this description is intended to include all such examples and their equivalents.

### BRIEF DESCRIPTION OF THE DRAWINGS

FIG. 1 is a schematic diagram of an example of a device for detecting air quality conditions in accordance with examples described herein.

FIG. 2 is a flow diagram of an example of detecting and/or reporting parameters related to air quality conditions in accordance with examples described herein.

FIG. 3 is a schematic diagram of an example of a computing device for performing functions described herein.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known components are shown in block diagram form in order to avoid obscuring such concepts.

Described herein are various examples related to detection and/or reporting of air quality parameter values in an indoor habitat. In an example, a device can include one or more sensors for sensing values of air quality parameters in a zone of the indoor habitat, possibly receiving other values of air quality parameters from other devices in other zones of the indoor habitat, and detecting air quality concerns based at least in part on the sensed values or other received values. Thus, in an example, the indoor habitat can be defined by the zones having corresponding devices for measuring and reporting air quality parameters, and as such air quality can be monitored in the habitat, and the device(s) can report parameter values and/or make decisions regarding triggering alarms, controlling smart devices in the habitat, etc., based on the air quality of its specific zone, a portion of the zones indicated as nearby or otherwise related, or of the habitat as a whole.

For example, the devices can communicate in a home network (e.g., over WiFi) and can each include sensors to measure and provide values of the air quality parameters to one another. In addition, the devices can be configured to report parameter values, trigger alarms, etc. to a controlling device, such as a mobile device of a user associated with the habitat, by communicating with the controlling device using the home network. In another example, the devices can be configured to report parameter values, provide instructions for operating other systems, etc., to the controlling device, such as a thermostat, where the thermostat may operate other systems (e.g., a heating, ventilation, and air conditioning (HVAC) system) based on the instructions or parameter values. Moreover, the devices can be configured, in one example, to provide local alarms based on detecting threshold values for the air quality parameters, detecting threshold values over a period of time for the air quality parameters, etc.

In an example, the sensors can measure air quality parameters such as pollen, dust, carbon monoxide, smoke, volatile organic compounds (VOCs), etc. In addition, for example, reporting or the parameters, triggering of alarms or commands to operate other systems, etc., may be based on determining whether the air quality parameters achieve certain thresholds. In one example, the device can measure the air quality parameters additionally considering air temperature or pressure, humidity, room occupancy, room light, etc., which can also be measured via the one or more sensors of the device. In this regard, habitat air quality management can be effectuated by using devices in each desired zone of the habitat to measure and report air quality parameters in this regard. The various thresholds may be configured for the device and may be specific to an individual occupying the habitat. As such, for example, an individual with asthma or other lung associated health issues may configure lower thresholds for the air quality parameters for triggering reporting or remedial action.

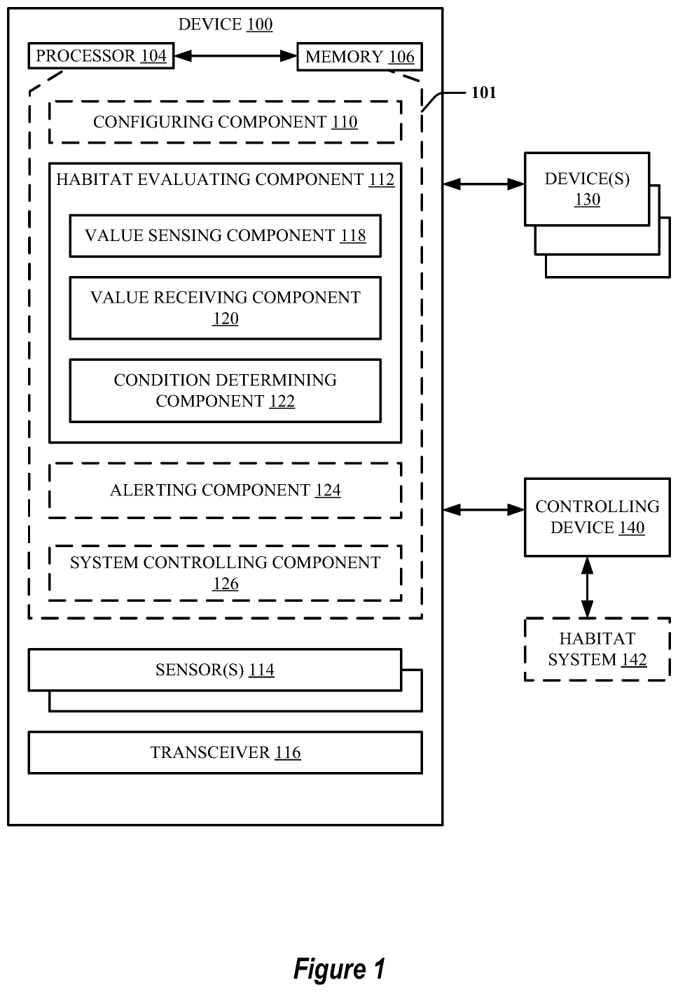

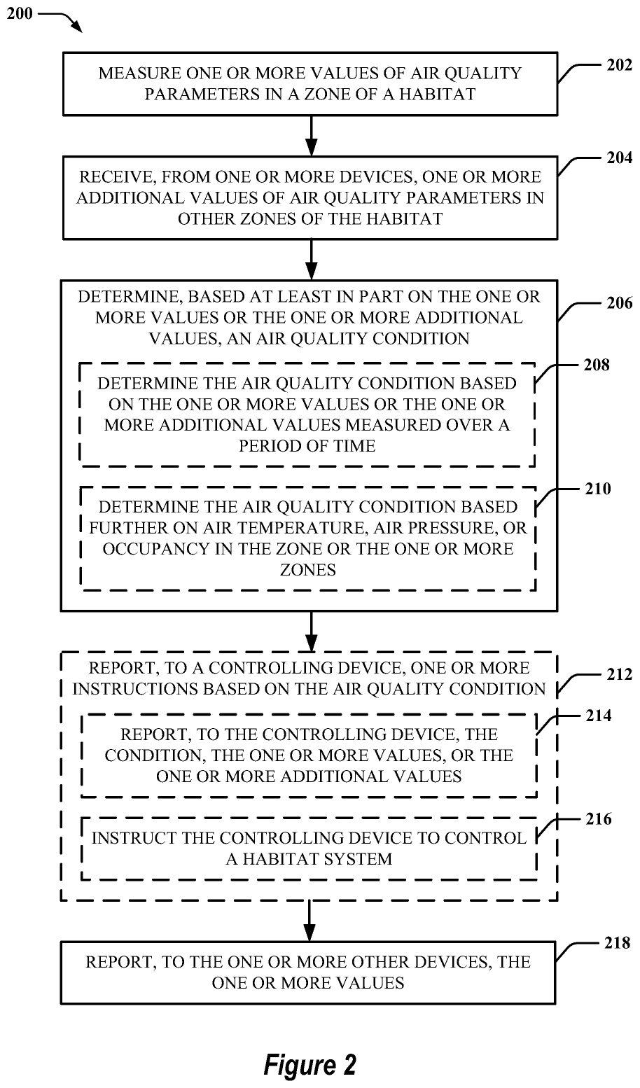

Turning now to FIGS. 1-3, examples are depicted with reference to one or more components and one or more methods that may perform the actions or operations described herein, where components and/or actions/operations in dashed line may be optional. Although the operations described below in FIG. 2 are presented in a particular order and/or as being performed by an example component, the ordering of the actions and the components performing the actions may be varied, in some examples, depending on the implementation. Moreover, in some examples, one or more of the following actions, functions, and/or described components may be performed by a specially-programmed processor, a processor executing specially-programmed software or computer-readable media, or by any other combination of a hardware component and/or a software component capable of performing the described actions or functions.

FIG. 1 is a schematic diagram of an example of a device 100 and/or related components for detecting and/or reporting air quality conditions in a habitat and/or related zone in accordance with aspects described herein. For example, device 100 can include or can otherwise be coupled with a processor 104 and/or memory 106, where the processor 104 and/or memory 106 can be configured to execute or store instructions or other parameters related to detecting and/or reporting air quality conditions, as described herein. For example, processor 104 and memory 106 may be separate components communicatively coupled by a bus (e.g., on a motherboard or other portion of a computing device, on an integrated circuit, such as a system on a chip (SoC), etc.), components integrated within one another (e.g., processor 104 can include the memory 106 as an on-board component 101), and/or the like. Memory 106 may store instructions, parameters, data structures, etc., for use/execution by processor 104 to perform functions described herein.

In an example, device 100 can optionally include a configuring component 110 for configuring a habitat evaluating component 112 and/or similar components of other devices 130 to measure and report parameters related to certain values of parameters measured by one or more sensors 114. Device 100 can also include the habitat evaluating component 112 configured to evaluate a habitat, or a certain zone thereof where the device 100 is installed, based on detecting measured air quality, temperature, pressure, humidity, room occupancy, or similar metrics measured by the one or more sensors 114. In this regard, device 100 can include the one or more sensors 114 that may comprise an air particulate sensor to measure certain air particulates, such as pollen or dust particles, etc. (e.g., as 2.5 microns or smaller in diameter). In another example, the one or more sensors 114 may include a sensor to measure VOCs, also referred to as total VOCs (TVOCs), which may be used to gauge levels of natural gas, and/or to calculate carbon dioxide (CO2) levels or eCO2 levels, which may be based on TVOCs, temperature, pressure, etc. In another example, the one or more sensors 114 may include a sensor to measure air temperature (e.g., a thermometer), a sensor to measure air pressure (e.g., a barometer), etc. In another example, the one or more sensors 114 may include a sensor to measure or determine motion or occupancy, etc. In another example, the one or more sensors 114 may include a sensor to measure certain gases (e.g., for carbon monoxide, alcohols, etc.), a sensor to measure smoke, etc. Device 100 can also include a transceiver 116 configured to communicate values of parameters measured by the sensors 114 and/or other sensors of other devices 130, to communicate parameters related to a detected air quality condition, which may include values of the parameters, corresponding alerts, commands to control a habitat system 142, and/or the like.

In an example, habitat evaluating component 112 can include a value sensing component 118 for receiving values of one or more parameters as measured by one or more sensors 114, a value receiving component 120 for receiving values of one or more parameters as received from one or more other devices 130 (e.g., based on sensor measurements at the one or more other devices 130), and/or a condition determining component 122 for detecting an air quality condition based on sensed and/or received values for one or more parameters related to air quality, temperature, pressure, humidity, room occupancy, etc., as described further herein. Device 100 can also optionally include an alerting component 124 for communicating an alert regarding a detected air quality condition, and/or a system controlling component 126 for controlling a habitat system 142 based on detecting the air quality condition.

In an example, device 100 can be deployed in a zone of a habitat, and similar devices 130 can be deployed in other zones of the habitat. The devices 100/130 can measure, via the one or more sensors 114, values of air quality parameters, such as pollen or dust count, carbon monoxide, smoke, VOCs, etc., and can communicate the values with one another (e.g., over a WiFi network). For example, a device 100 can be placed in each room of a building or in a portion of the rooms, etc., and can communicate with one another to obtain and/or provide a representation of conditions in the habitat defined by the rooms and/or building. In an example, the more devices 100 that are deployed throughout the building, the more accurate representation of the conditions in the building may be obtained, and thus the more accurate air quality condition report and/or corresponding remedial action taken.

Based on the values determined from the one or more sensors 114 (e.g., as determined by value sensing component 118 communicating with the sensors 114 over a bus or other medium) and/or the values received from other device(s) 130 (e.g., by value receiving component 120), condition determining component 122 can determine whether one or more air quality conditions exist in the zone or in other zones monitored by the other devices 130. The sensors 114 can work in parallel with one another to properly output data measurements around the device 100. As such a device 100/130 can be installed in each of various zones of the habitat to perform measurements in the corresponding zones. The devices 100/130 can communicate with one another via WiFi to create a wireless web cell that can interconnect the units and connect with the habitat system 142 and/or a corresponding control device 140.

In one example, condition determining component 122 can determine whether the one or more air quality conditions exist by comparing values of air quality parameters with configured or otherwise defined thresholds, comparing values of the air quality parameters with thresholds over a period of time, etc. For example, condition determining component 122 can perform data metric calculation and observation to justify condition of the environment and/or determine whether risks exist and/or take risk management actions, as described herein. In this regard, condition determining component 122 can consider not only a maximum value for the parameter being met, but can also measure the value over a period of time to detect a condition of prolonged exposure at lower levels, which can still be harmful for some conditions. Moreover, for example, condition determining component 122 can determine whether the one or more air quality conditions exist based on other parameters. For example, different thresholds can be defined for different other parameters, such as a higher threshold for pollen or dust for lower temperature or higher pressure measurements, higher threshold when the room is determined to not be occupied or having no detected motion for a threshold period of time, etc. Moreover, in an example, condition determining component 122 can determine different threshold based on other static parameters, such as room size, which can be configured and/or sensed via another sensor 114 that detects obstructions (e.g., walls, floor, ceiling, etc.) and distances thereto.

Where condition determining component 122 detects an air quality condition, alerting component 124 can trigger an alert of the condition and/or of values of the one or more parameters, an indication of the zone(s) within which the condition is detected, etc., to a controlling device 140. For example, the controlling device 140 may include a mobile device, an application executing thereon, a centralized home management device, etc. Alerting component 124 can send the alert via transceiver 116. The controlling device 140 may present the alert and/or a prompt to take a remedial action on a habitat system 142, such as shutting off ventilation in an occupied room, activating ventilation in a non-occupied room, opening one or more windows or other egress in a zone, etc. In another example, controlling device 140 may be configured to automatically take the remedial action. In another example, system controlling component 126 can send commands to the controlling device 140 to operate the habitat system 142 in this regard. In one example, controlling device 140 may include a thermostat that is able to receive control commands from various devices, such as device 100, and accordingly control habitat system 142. For example, habitat system 142 may include an HVAC system, as described.

As described, for example, values to be measured, corresponding thresholds, reporting triggers, remedial actions, etc. can be configured on the device 100. In an example, configuring component 110 can provide an interface for configuring these settings on the device and/or can allow for receiving a configuration from an application executing on another device, etc.

FIG. 2 is a flowchart of an example of a method 200 for detecting and reporting air quality conditions. For example, method 200 can be performed by the device 100, and is accordingly described with reference to FIG. 1, as a non-limiting example of an environment for carrying out method 200.

In method 200, at action 202, one or more values of air quality parameters in a zone of a habitat can be measured. In an example, value sensing component 118, e.g., in conjunction with processor 104, memory 106, habitat evaluating component 112, sensors 114, etc., can measure the one or more values of air quality parameters in the zone of the habitat. For example, value sensing component 118 can measure values of parameters in the zone within which the device 100 is installed and based on capabilities of the sensor(s) 114 configured in the device. As described, for example, value sensing component 118 may measure one or more of pollen or dust count, carbon monoxide, smoke, VOCs, air temperature, air pressure, humidity, occupancy, etc. In one example, the device 100 can be configured with zone information (e.g., via configuring component 110, control device 140, and/or the like), and the zone information may include an identifier of the zone, one or more other devices 130 that also operate to measure air quality parameters in the same zone, and/or the like.

In method 200, at action 204, one or more additional values of air quality parameters in other zones of the habitat can be received from one or more other devices. In an example, value receiving component 120, e.g., in conjunction with processor 104, memory 106, habitat evaluating component 112, transceiver 116, etc., can receive, from one or more other devices, the one or more additional values of air quality parameters in the other zones of the habitat. For example, value receiving component 120 can receive values of similar air quality parameters as those sensed by sensor(s) 114, but in the other zones of the habitat. In this regard, the devices 100/130 can communicate values of the air quality parameters with one another and can each make decisions regarding air quality conditions based on input from one another and/or based on knowing within which zones the devices are located (and/or an indication of nearby or related zones, etc.). In one example, value receiving component 120 can determine whether received values are for the same zone. For example, value receiving component 120 can determine whether the received values are from devices 130 operating in the same zone, which can be based on determining a zone identifier associated with the zone as indicated in the received values, determining that the other device 130 from which the values are received is associated with the same zone (e.g., based on receiving a list of devices in the zone), etc.

In method 200, at action 206, an air quality condition can be determined based at least in part on the one or more values or the one or more additional values. In an example, condition detecting component 122, e.g., in conjunction with processor 104, memory 106, habitat evaluating component 112, etc., can determine, based at least in part on the one or more values or the one or more additional values, the air quality condition. For example, condition detecting component 122 can determine the air quality condition based on determining that the one or more values or one or more additional values of air quality parameters achieve one or more thresholds (e.g., pollen or dust count achieves a threshold, carbon monoxide level achieves a threshold, etc.), a combination (e.g., average) of the one or more values or one or more additional values of air quality parameters achieve one or more thresholds, etc.

In determining the air quality condition at action 206, optionally at action 208, the air quality condition can be determined based on the one or more values or the one or more additional values measured over a period of time. In an example, condition detecting component 122, e.g., in conjunction with processor 104, memory 106, habitat evaluating component 112, etc., can determine the air quality condition based on the one or more values or the one or more additional values measured over the period of time. In this regard, as described, condition detecting component 122 can detect exposure to harmful air quality conditions at lower levels over periods of time (e.g., prolonged exposure over longer periods of time, such as several hours, days, weeks, etc.), which may be as harmful or more harmful than the air quality condition achieving a higher threshold at an instance in time. For example, the various levels, periods of time, etc. can be configured via configuring component 110 and/or specified via an interface and/or the like, as described herein.

In another example, in determining the air quality condition at action 206, optionally at action 210, the air quality condition can be determined based further on air temperature, air pressure, or occupancy in the zone or the one or more zones. In an example, condition detecting component 122, e.g., in conjunction with processor 104, memory 106, habitat evaluating component 112, etc., can determine the air quality condition based further on air temperature, air pressure, or occupancy in the zone or the one or more zones. For example, condition detecting component 122 can determine different thresholds for the air quality parameters for different air temperatures, air pressures, and/or whether the zone is occupied or motion is detected in the zone. In another example detecting particles in the air may be more harmful at higher temperatures, and thus thresholds for the air quality parameters may be mapped to temperature. Additionally, as described, condition detecting component 122 may consider the air quality condition as a combination (e.g., average) of values reported or measured by various devices in a zone, may consider the temperature or occupancy in a zone based on a combination (e.g., average) of values reported or measured by various devices in the zone, etc.

In any case, for example, condition detecting component 122 can determine an air quality index (AQI) value for a particular zone and/or for the habitat (e.g., by combining parameter values determined by various devices 100 deployed in the habitat). In an example, condition detecting component 122 can determine the AQI value using calculation of metrics that may be specified by an agency, such as a governmental agency (e.g., environmental protection agency (EPA) in the United States, etc.). In this example, condition detecting component 122 can be configured with parameters for computing the AQI based on parameter values measured for one or more air quality parameters by one or more devices 100 in the habitat.

In method 200, optionally at action 212, one or more instructions can be reported to a controlling device based on the air quality condition. In an example, alerting component 124 and/or system controlling component 126, e.g., in conjunction with processor 104, memory 106, etc., can report, to the controlling device 140, one or more instructions based on the air quality condition.

In one example, in reporting the instructions at action 212, optionally at action 214, the condition, the one or more values, or the one or more additional values can be reported to the controlling device. In an example, alerting component 124, e.g., in conjunction with processor 104, memory 106, etc., can report, to the controlling device 140, the condition, the one or more values, or the one or more additional values. In one example, the one or more values or one or more additional values as reported may include a AQI value. In this regard, the controlling device 140 can be alerted of the condition and/or associated values (e.g., and the corresponding zones with values exceeding thresholds). In one example, this may include an offer of suggestion about the use of the habitat system 142, suggestion to call an emergency number (e.g., 911) in the case of values that achieve or exceed a threatening threshold level, etc. The controlling device 140 may take action on a habitat system 142 based on the alert, initiate a call or other communication to an emergency number, etc. In one example, alerting component 124 can output an interface that indicates the values or additional values, and may include an output of the computation data over the whole habitat (e.g., as received and/or modified from multiple devices 100), which may include the AQI value, temperature, pressure, humidity, etc.

In another example, in reporting the instructions at action 212, optionally at action 216, the controlling device can be instructed to control the habitat system. In an example, system controlling component 126, e.g., in conjunction with processor 104, memory 106, etc., can instruct the controlling device to control the habitat system. For example, system controlling component 126 can instruct the controlling device 140 to start ventilation (e.g., to purge air of pollutants for easier breathing quality) or stop ventilation (e.g., to prevent from spreading or further activating pollutants) of the habitat system 142 (e.g., which may also depend on detected room occupancy), open a window or other egress, start or stop a humidifier or dehumidifier in the zone (e.g., based on detected humidity), etc. where the controlling device 140 allow for receiving such commands and accordingly operating the habitat system 142. In an example, in this regard, where HVACs are otherwise setup and ran for maximum occupancy of a space, system controlling component 126 can use occupancy information to regulate HVAC based on quantity to decrease cost and environmental impact of the HVAC.

In method 200, at action 218, the one or more values can be reported to the one or more other devices. In an example, value sensing component 118, e.g., in conjunction with processor 104, memory 106, habitat evaluating component 112, transceiver 116, etc., can report, to the one or more other devices, the one or more values. As described, this can enable the other devices 130 to similarly determine air quality conditions in the habitat based on a collection or combination of values.

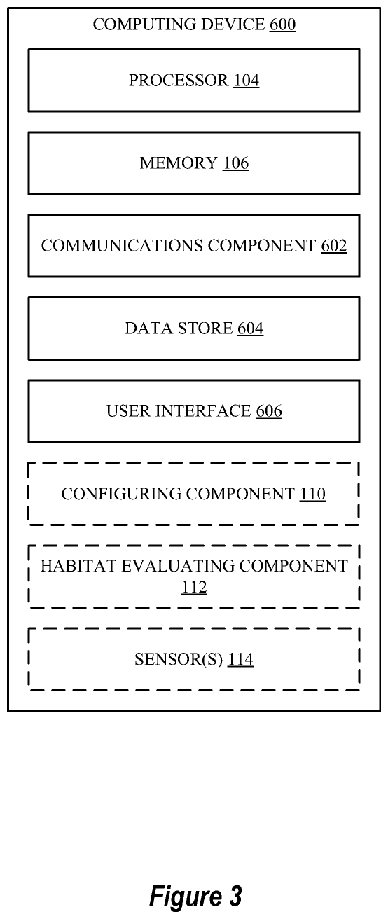

FIG. 3 illustrates an example of computing device 300 including additional optional component details as those shown in FIG. 1. In one example, computing device 300 may include processor 104 for carrying out processing functions associated with one or more of components and functions described herein. Processor 104 can include a single or multiple set of processors or multi-core processors. Moreover, processor 104 can be implemented as an integrated processing system and/or a distributed processing system.

Computing device 300 may further include memory 106, such as for storing local versions of applications being executed by processor 104, related instructions, parameters, etc. Memory 106 can include a type of memory usable by a computer, such as random access memory (RAM), read only memory (ROM), tapes, magnetic discs, optical discs, volatile memory, non-volatile memory, and any combination thereof. Additionally, processor 104 and memory 106 may include and execute a configuring component 110, habitat evaluating component 112, and/or components thereof, etc., as described herein, and/or other components of the computing device 300.

Further, computing device 300 may include a communications component 302 that provides for establishing and maintaining communications with one or more other devices, parties, entities, etc. utilizing hardware, software, and services as described herein. Communications component 302 may carry communications between components on computing device 300, as well as between computing device 300 and external devices, such as devices located across a communications network and/or devices serially or locally connected to computing device 300. For example, communications component 302 may include one or more buses, and may further include transmit chain components and receive chain components associated with a wireless or wired transmitter and receiver, respectively, operable for interfacing with external devices. For example, communications component 302 can carry communications between configuring component 110, habitat evaluating component 112, sensor(s) 114, etc., one or more of which may be executing on another device (or the same device), etc. In addition, communications component 302 may include a transceiver 116, as described herein.

Additionally, computing device 300 may include a data store 304, which can be any suitable combination of hardware and/or software, that provides for mass storage of information, databases, and programs employed in connection with examples described herein. For example, data store 304 may be or may include a data repository for applications and/or related parameters not currently being executed by processor 104. In addition, data store 304 may be a data repository for configuring component 110, habitat evaluating component 112, and/or components thereof, etc. executing on the processor 104, and/or one or more other components of the computing device 300.

Computing device 300 may also include a user interface component 306 operable to receive inputs from a user of computing device 300 and further operable to generate outputs for presentation to the user. User interface component 306 may include one or more input devices, including but not limited to a keyboard, a number pad, a mouse, a touch-sensitive display, a navigation key, a function key, a microphone, a voice recognition component, a gesture recognition component, a depth sensor, a gaze tracking sensor, any other mechanism capable of receiving an input from a user, or any combination thereof. Further, user interface component 306 may include one or more output devices, including but not limited to a speaker, a haptic feedback mechanism, a printer, any other mechanism capable of presenting an output to a user, or any combination thereof.

Computing device 300 can also include one or more of a configuring component 110 for configuring parameters for a habitat evaluating component 112, as described above, and/or may include the habitat evaluating component 112 for determining air quality conditions in the habitat or related zones, as described above.

By way of example, an element, or any portion of an element, or any combination of elements may be implemented with a “processing system” that includes one or more processors. Examples of processors include microprocessors, microcontrollers, digital signal processors (DSPs), field programmable gate arrays (FPGAs), programmable logic devices (PLDs), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software modules, applications, software applications, software packages, routines, subroutines, objects, executables, threads of execution, procedures, functions, etc., whether referred to as software, firmware, middleware, microcode, hardware description language, or otherwise.

Accordingly, in one or more examples, one or more of the functions described may be implemented in hardware, software, firmware, or any combination thereof. If implemented in software, the functions may be stored on or encoded as one or more instructions or code on a computer-readable medium. Computer-readable media includes computer storage media. Storage media may be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer. Disk and disc, as used herein, includes compact disc (CD), laser disc, optical disc, digital versatile disc (DVD), and floppy disk where disks usually reproduce data magnetically, while discs reproduce data optically with lasers. Combinations of the above should also be included within the scope of computer-readable media.

The previous description is provided to enable any person skilled in the art to practice the various examples described herein. Various modifications to these examples will be readily apparent to those skilled in the art, and the generic principles defined herein may be applied to other examples. Thus, the claims are not intended to be limited to the examples shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. All structural and functional equivalents to the elements of the various examples described herein that are known or later come to be known to those of ordinary skill in the art are intended to be encompassed by the claims. Moreover, nothing disclosed herein is intended to be dedicated to the public regardless of whether such disclosure is explicitly recited in the claims. No claim element is to be construed as a means plus function unless the element is expressly recited using the phrase “means for.”

What is claimed is:

1. A device for reporting air quality parameters for an environment, comprising:
a sensor configured to measure a first value of an air quality parameter in a first zone of the environment;
a transceiver configured to receive a second value of the air quality parameter in a second zone of the environment; and
a processor configured to:
determine, based on the first value and the second value, an air quality condition.

2. The device of claim 1, wherein the processor is further configured to report, to a controlling device and via the transceiver, an instruction based on the air quality condition.

3. The device of claim 2, wherein the controlling device includes a monitoring application, and wherein reporting the instruction comprises reporting the air quality condition, the first value, or the second value to the controlling device.

4. The device of claim 2, wherein the controlling device includes a thermostat, and wherein reporting the instruction comprises transmitting a command to the thermostat to modify operation of a heating, ventilation, and air conditioning (HVAC) system based on the air quality condition.

5. The device of claim 1, wherein the sensor is configured to measure the first value of the air quality parameter over a period of time, and wherein the processor is configured to determine the air quality condition based on evaluating the first value of the air quality parameter over the period of time.

6. The device of claim 1, wherein the sensor is configured to measure at least one of a carbon monoxide count, a pollen count, or a volatile organic compound (VOC) count.

7. The device of claim 1, wherein the processor is configured to determine the air quality condition based on considering the first value or the second value in conjunction with a room temperature or pressure measured by the sensor.

8. The device of claim 1, wherein the processor is configured to determine the air quality condition based on considering the first value or the second value in conjunction with an occupancy of the first zone or the second zone.

9. The device of claim 1, wherein the transceiver is configured to communicate over a WiFi network.

10. The device of claim 1, wherein the processor is further configured to report, to another device via the transceiver, the first value.

11. A computer-implemented method for reporting air quality parameters for an environment, comprising:
measuring, via a sensor, a first value of an air quality parameter in a first zone of the environment;
receiving a second value of the air quality parameter in a second zone of the environment; and
determining, based on the first value and the second value, an air quality condition.

12. The computer-implemented method of claim 11, further comprising reporting, to a controlling device, an instruction based on the air quality condition.

13. The computer-implemented method of claim 12, wherein the controlling device includes a monitoring application, and wherein reporting the instruction comprises reporting at least one of the air quality condition, the first value, or the second value to the controlling device.

14. The computer-implemented method of claim 12, wherein the controlling device includes a thermostat, and wherein reporting the instruction comprises transmitting a command to the thermostat to modify operation of a heating, ventilation, and air conditioning (HVAC) system based on the air quality condition.

15. The computer-implemented method of claim 11, wherein the sensor is configured to measure the first value of the air quality parameter over a period of time, and wherein determining the air quality condition is based on evaluating the first value of the air quality parameter over the period of time.

16. The computer-implemented method of claim 11, wherein the sensor is configured to measure at least one of a carbon monoxide count, pollen count, or a volatile organic compound (VOC) count.

17. The computer-implemented method of claim 11, wherein determining the air quality condition is based on considering at least one of the first value or the second value in conjunction with a room temperature or pressure measured by the sensor.

18. The computer-implemented method of claim 11, wherein determining the air quality condition is based on considering at least one of the first value or the second value in conjunction with an occupancy of the first zone or the second zone.

19. The computer-implemented method of claim 11, wherein receiving the second value is over a WiFi network.

20. The computer-implemented method of claim 11, further comprising reporting, to another device, the first value.

21. A non-transitory computer-readable medium, comprising code executable by a processor for reporting air quality parameters for an environment, the code comprising code for:
measuring, via a sensor, a first value of an air quality parameter in a first zone of the environment;
receiving a second value of the air quality parameter in a second zones of the environment; and
determining, based on the first value and the second value, an air quality condition.

22. The non-transitory computer-readable medium of claim 21, wherein the code further comprises code for reporting, to a controlling device, an instruction based on the air quality condition.

23. The non-transitory computer-readable medium of claim 22, wherein the controlling device includes a monitoring application, and wherein the code for reporting the instruction reports at least one of the air quality condition, the first value, or the second value to the controlling device.
